(19) United States
(12) Patent Application Publication
(10) Pub. No.: US 2003/0026773 A1
(43) Pub. Date: Feb. 6, 2003

(54) Anhydrous liquid antiperspirant emulsions

(75) Inventors: David Frederick Swaile, Cincinnati, OH (US); Amy Michelle Capretta, Cincinnati, OH (US); Jennifer Elaine Hilvert, Cincinnati, OH (US); Cheryl Marie Williams, Fairfield, OH (US); Timothy James Welch, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 09/891,968

(22) Filed: Jun. 26, 2001

(51) Int. Cl. A61K 7/32; A61K 7/34; A61K 7/38; A61K 7/00

(52) U.S. Cl. 424/65; 424/66; 424/68; 424/400; 424/401

(57) ABSTRACT

Disclosed are liquid anhydrous antiperspirant emulsions, comprising (a) an external phase containing a non-volatile hydrophobic liquid; and (b) an internal phase containing an antiperspirant active solubilized in a polar solvent; wherein the emulsions have an external to internal phase ratio of at least about 1.25 and a weight ratio of the non-volatile hydrophobic liquid to the combination of antiperspirant active and polar solvent in the internal phase of at least about 0.33. The anhydrous antiperspirant emulsions provide little or no visible residue on the skin and also provide for a smooth, dry, non-sticky, skin-feel during and after application. These emulsions are especially useful when delivered from a roll-on applicator.

### TECHNICAL FIELD

The present invention relates to anhydrous liquid antiperspirant emulsions containing solubilized antiperspirant active.

### BACKGROUND OF THE INVENTION

There are many different liquid antiperspirant formulations known for use in controlling or inhibiting underarm perspiration and odor. These formulations are most typically in the form of roll-ons or pressurized aerosols, although a number of less conventional liquid formulations are also known. These formulations typically contain an antiperspirant active such as an aluminum salt, or a combination of an aluminum and zirconium salt, that is dissolved or suspended in a suitable anhydrous or aqueous carrier. It is well known that these antiperspirant liquid formulations can be formulated in a variety of liquid product forms, including suspensions, solutions and emulsions.

Among the various liquid antiperspirant formulations described in the literature or otherwise made available to consumers, the emulsion form in particular has fallen out of favor in some markets due largely to formulation limitations and poor application cosmetics. These liquid antiperspirant emulsions are often excessively sticky and have an undesirably wet feel during and after application. Although these poor application cosmetics can be reduced by the use of an anhydrous rather than an aqueous carrier in the emulsion, they still tend to feel excessively sticky during and after application.

It has now been found that antiperspirant emulsions can be prepared that provide a dry, smooth, non-sticky skin feel during and after application to the underarm. It has been found that such benefits can be realized from an anhydrous, liquid, antiperspirant emulsion, provided that the emulsion comprises (a) an external phase containing a non volatile hydrophobic liquid; and (b) an internal phase containing an antiperspirant active solubilized in a polar solvent; wherein the anhydrous antiperspirant emulsion has an external to internal phase ratio of at least about 1.25 and a weight ratio of the non-volatile hydrophobic liquid to the combination of antiperspirant active and polar solvent in the internal phase of at least about 0.33.

It has been found that the anhydrous liquid antiperspirant emulsions of the present invention can be formulated to provide little or no visible residue on the skin, as well as provide improved application cosmetics as compared to many other known antiperspirant emulsions. It has been found that such benefits can be delivered from an anhydrous antiperspirant emulsion as described herein, provided that the emulsion has the requisite phase ratio and the requisite weight ratio of non-volatile hydrophobic liquid to solubilized antiperspirant active and polar solvent.

### SUMMARY OF THE INVENTION

The present invention is directed anhydrous liquid antiperspirant emulsions, comprising (a) an external phase containing a non-volatile hydrophobic liquid; and (b) an internal phase containing an antiperspirant active solubilized in a polar solvent; wherein the anhydrous antiperspirant emulsions have an external to internal phase ratio of at least about 1.25 and a weight ratio of the non-volatile hydrophobic liquid to the combination of antiperspirant active and polar solvent in the internal phase of at least about 0.33.

It has been found that an antiperspirant emulsion can be formulated to provide little or no visible residue on the skin, and also to provide a smoother, drier skin feel during and after topical application as compared to many other known antiperspirant emulsions. It has been found that such benefits can be delivered from an anhydrous antiperspirant emulsion as described herein, provided that the emulsion has the requisite phase ratios and the requisite weight ratio of non volatile liquid to solubilized antiperspirant active and polar solvent.

### DETAILED DESCRIPTION OF THE INVENTION

The anhydrous antiperspirant emulsions of the present invention comprise an external, hydrophobic phase containing a non-volatile hydrophobic liquid, and an internal polar-phase containing antiperspirant active solubilized by a polar solvent. These and other essential elements or limitations of the antiperspirant emulsions of the present invention are described in detail hereinafter.

The term “anhydrous” as used herein, unless otherwise specified, refers to the emulsion compositions of the present invention, as well as any other referenced composition, material, or ingredient containing less than about 15%, more preferably less than about 5%, even more preferably less than about 1%, even more preferably zero percent, by weight of water.

The term “ambient conditions” as used herein, unless otherwise specified, refers to surrounding conditions at about one atmosphere of pressure, at about 50% relative humidity, and at about 25° C.

The term “non-volatile” as used herein, unless otherwise specified, refers to those materials having no significant or measurable vapor pressure as measured at 25° C., which typically includes those materials having a vapor pressure less than about 0.01 mmHg. Such non-volatile materials will generally have a viscosity as measured under ambient conditions of at least about 10 centistokes. The term “volatile” as used herein, unless otherwise specified, therefore refers to all other materials that do not satisfy the above-described characteristics of a non-volatile material.

The term “phase ratio” as used herein, unless otherwise specified, refers to a weight percentage of the materials in the external hydrophobic phase divided by the weight percentage of materials in the internal polar phase (including antiperspirant active and polar solvent). The weight percentage of any emulsifying agents should be included in the phase in which it is most soluble. In this context, each of the weight percentages are by weight of the antiperspirant emulsion composition.

All percentages, parts and ratios as used herein, unless otherwise specified, are by weight of the total composition, i.e., the antiperspirant emulsion composition. All such weights as they pertain to listed ingredients are based on the active level and, therefore, do not include solvents or by-products that may be included in commercially available materials, unless otherwise specified.

The anhydrous antiperspirant emulsions of the present invention can comprise, consist of, or consist essentially of the essential elements and limitations of the invention described herein, as well as any additional or optional ingredients, components, or limitations described herein or otherwise useful in antiperspirant applications.

### Anhydrous Emulsions

The liquid antiperspirant emulsions of the present invention are in the form of multi-phase emulsions, typically a two-phase emulsion, wherein the emulsion has an internal, discontinuous, polar-phase containing solubilized antiperspirant active in a polar solvent, and a continuous, hydrophobic, external-phase containing a suitable hydrophobic non-volatile liquid.

The multiple phases of the antiperspirant emulsions are formulated so that an internal phase comprises antiperspirant active solubilized in an anhydrous polar solvent, the polar solvent and the antiperspirant active being described in greater detail hereinafter. These emulsions are also formulated so that a continuous external-phase of the emulsion comprises a non-volatile hydrophobic liquid, most typically a dimethicone liquid, all within a hydrophobic liquid matrix. The hydrophobic liquid for use in the external-phase of the emulsion is described in greater detail hereinafter.

It has been found that the antiperspirant emulsions of the present invention must have an internal to external phase ratio as defined herein of at least about 1.25, preferably from about 1.25 to about 5.0, more preferably from about 1.7 to about 3.5.

It has also been found that the antiperspirant emulsions of the present invention must have a weight ratio of the hydrophobic liquid to the combination of solubilized antiperspirant active and polar solvent of at least about 0.33, preferably from about 0.5 to about 5.0, even more preferably from about 0.5 to about 1.5.

The antiperspirant emulsions of the present invention must also be anhydrous liquids, so that the internal and external phases of the emulsions are also anhydrous, although even these anhydrous phases may contain small amounts of water associated with the antiperspirant active during formulation. These emulsions are contained within a suitable container or applicator and are preferably in the form of a visibly clear or translucent liquid.

The internal and external phases of the emulsions may further comprise any of a variety of materials, so long as such materials are compatible with the emulsion phase in which that optional material is formulated, and provided that the phase ratios and other weight ratios as defined herein are maintained within the specified range. For example, the internal phase may further comprise any of a variety of relatively polar materials such as monohydric alcohols or other materials having relatively polar functional groups, whereas the external hydrophobic phase may further comprise any of a variety of relatively hydrophobic materials suitable for use in personal care products such as volatile silicone or non-silicone liquids, various dispersed solids, and so forth. The external phase preferably further comprises a volatile silicone material such as cyclopentasiloxane.

### Solubilized Antiperspirant Active

The anhydrous antiperspirant emulsions of the present invention comprise an internal phase containing a solubilized antiperspirant active suitable for application to human skin. The concentration of antiperspirant active in the composition should be sufficient to provide the finished antiperspirant product with the desired perspiration wetness and odor control. The antiperspirant active may be solubilized by any anhydrous polar solvent suitable for application to the skin that is otherwise compatible with the selected ingredients and product specifications of the anhydrous emulsion.

Solubilized antiperspirant active concentrations in the antiperspirant emulsions preferably range from about 0.1% to about 26%, more preferably from about 1% to about 20%, even more preferably from about 2% to about 10%, by weight of the composition. All such weight percentages are calculated on an anhydrous metal salt basis exclusive of water and any complexing or buffering agent such as glycine, glycine salts, or other complexing or buffering agent.

The solubilized antiperspirant active for use in the antiperspirant emulsions of the present invention include any compound, composition or other material having antiperspirant activity. Preferred antiperspirant actives include astringent metallic salts, especially the inorganic and organic salts of aluminum, zirconium and zinc, as well as mixtures thereof. Particularly preferred are salts such as aluminum halides, aluminum chlorohydrate, aluminum hydroxyhalides, zirconyl oxyhalides, zirconyl hydroxyhalides, and mixtures thereof.

Preferred aluminum salts for use in the antiperspirant emulsions include those that conform to the formula:

Al₂(OH)ₐCl_{b}.xH₂O

wherein a is from about 2 to about 5; the sum of a and b is about 6; x is from about 1 to about 6; and wherein a, b, and x may have non-integer values. Particularly preferred are the aluminum chlorhydroxides referred to as “5/6 basic chlorhydroxide”, wherein a=5, and “2/3 basic chlorhydroxide” wherein a=4. Processes for preparing aluminum salts are disclosed in U.S. Pat. No. 3,887,692, Gilman, issued Jun. 3, 1975; U.S. Pat. No. 3,904,741, Jones et al., issued Sep. 9, 1975; U.S. Pat. No. 4,359,456, and Gosling et al., issued Nov. 16, 1982, all of which are incorporated herein by reference. Mixtures of aluminum salts are described in British Patent Specification 1,347,950, Shin et al., published Feb. 27, 1974.

Preferred zirconium salts for use in the antiperspirant emulsions include those that conform to the formula:

ZrO(OH)₂₋ₐClₐ.xH₂O

wherein a is any number having a value of from 0 to about 2; x is from about 1 to about 7; and wherein a and x may both have non-integer values. Preferred zirconium salts are those complexes which additionally contain aluminum and glycine, commonly known as ZAG complexes. These ZAG complexes contain aluminum chlorhydroxide and zirconyl hydroxy chloride conforming to the above described formulas. Such ZAG complexes are described in U.S. Pat. No. 3,679,068, Luedders et al., issued Feb. 12, 1974; Great Britain Patent Application 2,144,992, Callaghan et al., published Mar. 20, 1985; and U.S. Pat. No. 4,120,948, Shelton, issued Oct. 17, 1978.

Preferred antiperspirant actives for use in the emulsions compositions herein include aluminum chlorohydrate, aluminum dichlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum zirconium trichlorohydrate, aluminum zirconium tetrachlorohydrate, aluminum zirconium pentatchlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, aluminum chloride, aluminum sulfate buffered, and combinations thereof.

Non limiting examples of solubilized antiperspirant active for use in the antiperspirant emulsions of the present invention, and methods of making the solubilized active, are described in U.S. Pat. No. 6,149,897 (Swaile); U.S. Pat. No. 6,126,928 (Swaile); and U.S. Pat. No. 5,968,489 (Swaile et al.), which descriptions are incorporated herein by reference. Other non limiting examples of solubilized antiperspirant active and methods of making it are described in EP 0 404 533 (Smith et al.).

### Polar Solvent

The antiperspirant emulsions of the present invention comprise a polar solvent for solubilizing or helping to solubilize the antiperspirant active material within the internal-phase of the antiperspirant emulsion. The concentration of the polar solvent generally ranges from about 1% to about 45%, more preferably from about 5% to about 35%, by weight of the emulsion, but must also be selected so that the weight ratio of the non-volatile hydrophobic fluid to the antiperspirant active and polar solvent falls within the range as defined herein.

The term “polar solvent” as used herein, unless otherwise specified, refers to any liquid that can solubilize or help to solubilize the antiperspirant active within the internal liquid phase of the liquid antiperspirant emulsion, or that is otherwise soluble or miscible in the internal phase of the antiperspirant emulsion wherein the internal phase contains solubilized antiperspirant active. The polar solvent will generally have a solubility parameter of greater than about 10 cal/cm³ while the hydrophobic liquid as described herein will generally have a solubility parameter of less than about 10 cal/cm³, although it is possible for purposes of the present invention to have a polar solvent with a solubility parameter of less than about 10 cal/cm³ and a hydrophobic liquid with a solubility parameter of greater than about 10 cal/cm³. It is also possible for purposes of the present invention for a solvent in one embodiment to be a polar solvent that partitions into the internal polar phase, and for other embodiments the same liquid could then be part of and partition into the external hydrophobic phase. Solubility parameters and means for determining them are well known in the chemical arts, examples of which are described by C. D. Vaughan, “Solubility Effects in Product, Package, Penetration and Preservation” 103 Cosmetics and Toiletries, 47-49, October 1988: and C.D. Vaughan, “Using Solubility Parameters in Cosmetics Formulations”, 36 J. Soc. Cosmetic Chemists 319-333, September/October, 1988.

The polar solvent for use in the antiperspirant emulsions of the present invention is preferably a liquid polyol having two or more hydroxyl groups, preferably a polyol having at least 3 carbon atoms and adjacent hydroxy-substituted carbon atoms at the α and β positions of the liquid polyol. Preferred liquid polyols for use in the compositions are those that conform to the formula:

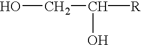

wherein R is an amide, ester, alkyl, ether or silicone-containing moiety, each moiety containing at least one carbon atom. The R group is preferably an alkyl or ether group, more preferably an alkyl group having from about 1 to about 10 carbon atoms, more preferably from about 4 to about 6 carbon atoms. The liquid polyols preferably have either 2 or 3 hydroxyl groups in total.

The R group on the liquid polyol can be substituted or unsubstituted, branched or straight or cyclic, saturated or unsaturated. Non limiting examples of suitable substituents include hydroxyl groups, amines, amides, esters, ethers, alkoxylate groups (e.g., ethoxylates, propoxylates, etc.) and so forth.

Non limiting examples of suitable polar solvent for use in the antiperspirant emulsions of the present invention include 1,2-butanediol; 1,2-pentanediol; 4-methyl-1,2-pentanediol; 2-methyl-1,2-pentanediol; 3,3-methyl-1,2-butanediol; 4-methyl-1,2-hexanediol; 1,2-heptanediol; 3-phenyl-1,2-propanediol; 1,2,6-hexanetriol; 1,2-hexandiol; 1,2,4-butanetriol; propylene glycol; glycerin; sorbitol; and combinations thereof. Other suitable liquid polyols include glycerol ethers such as glycerol isopropyl ether; glycerol propyl ether; glycerol ethyl ether; glycerol methyl ether; glycerol butyl ether; glycerol isopentyl ether; diglycerol isopropyl ether; diglycerol isobutyl ether; diglycerol; triglycerol; triglycerol ; isopropyl ether; and combinations thereof.

Still other suitable polar solvents include acetic acid glycerol ester; propanoic acid glycerol ester; butanoic acid glycerol ester; 3-methyl butanoic acid glycerol ester; and 3-trimethylsily-1,2-propane diol; silicone-containing 1, 2-diols such as those described in U.S. Pat. No. 5,969,172 (Nye); and combinations thereof.

Especially preferred are those embodiments having a polar solvent that contains glycerin in combination with at least one other polar solvent, preferably glycerin in combination with a 1,2-polyol solvent as described herein.

These polar solvents may be used in combination with other anhydrous liquids, provided that such other liquids are suitable for topical application to the skin and are compatible with the selected emulsion system and the ingredients therein. It should be noted, however, that as such other liquids become more hydrophobic and less polar, that such materials will instead be formulated into or otherwise partition to the external hydrophobic phase of the antiperspirant emulsions.

The polar solvent may comprise highly polar liquids such as ethanol or other similar solvents, but only to the extent that such solvents can be formulated into the composition without unduly affecting the ability of the emulsion to deliver active to the skin during application. The antiperspirant emulsion and therefore the polar solvent in the emulsion are preferably substantially free of ethanol. In this context, the term “substantially free” means that the antiperspirant emulsion preferably contains less than 20%, more preferably less than 10%, even more preferably less than 3%, most preferably zero percent, of ethanol by weight of the emulsion composition.

### External Phase Hydrophobic Liquid

The external hydrophobic phase of the antiperspirant emulsions of the present invention comprises a non-volatile hydrophobic liquid. The hydrophobic liquid can be any volatile silicone or non-silicone containing fluid that is liquid at room temperature or otherwise liquid within the finished emulsion formulation, and is compatible with the emulsion system and the ingredients therein, and does not otherwise partition significantly into the internal polar phase of the emulsion system.

The term “hydrophobic” as used herein, unless otherwise specified, refers to those materials that are contained within or primarily partition into the external phase of the emulsion compositions herein.

The non-volatile hydrophobic liquid suitable for use in the external phase of the antiperspirant emulsion is soluble in or miscible with the external hydrophobic phase of the antiperspirant emulsions herein. These non-volatile hydrophobic liquids can be cyclic or linear or branched chain, substituted or unsubstituted, silicone or non-silicone containing. The non-volatile hydrophobic liquid may further comprise volatile hydrophobic liquids in combination with the non-volatile liquid.

The concentration of the non-volatile hydrophobic liquid in the antiperspirant emulsions generally ranges from about 5% to about 70%, more preferably from about 8% to about 45%, even more preferably from about 10% to about 20%, by weight of the liquid antiperspirant emulsion, although specific non-volatile hydrophobic fluid concentrations must still be selected so that the ratio of the silicone fluid to the combination of antiperspirant active and polar solvent falls within the requisite range.

Non limiting examples of suitable volatile silicones as hydrophobic fluids for optional use herein, and which will typically be part of the external hydrophobic phase, are described in Todd et al., “Volatile Silicone Fluids for Cosmetics”, Cosmetics and Toiletries, 91:27-32 (1976), which descriptions are incorporated herein by reference. Preferred among these volatile silicones are the cyclic silicones having from about 3 to about 7, more preferably from about 4 to about 5, silicon atoms. Most preferably are those that conform to the formula:

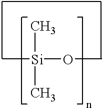

wherein n is from about 3 to about 7, preferably from about 4 to about 5, most preferably 5. These volatile cyclic silicones generally have a viscosity value of less than about 10 centistokes as measured at 25° C. Suitable volatile silicones for use herein include, but are not limited to, Cyclomethicone D-5 (commercially available from G. E. Silicones); DC 1184, DC 1731, DC 344, and DC 345 (commercially available from Dow Corning Corp.); GE 7207, GE 7158 and Silicone Fluids SF-1202 and SF-1173 (available from General Electric Co.); SWS-03314, SWS-03400, F-222, F-223, F-250, F-251 (available from SWS Silicones Corp.); Volatile Silicones 7158, 7207, 7349 (available from Union Carbide); Masil SF-V (available from Mazer) and combinations thereof. Cyclopentasiloxane is most preferred among the volatile silicone liquids.

Non-limiting examples of non-volatile silicone liquids as hydrophobic fluids for use in the antiperspirant emulsions of the present invention include those that conform to either of the formulas:

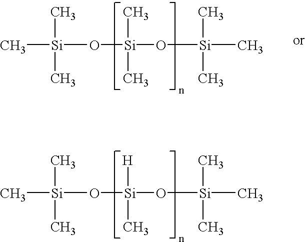

wherein n is greater than or equal to 1. These linear silicone materials will generally have viscosity values of from about 10 centistoke to about 100,000 centistoke, preferably less than about 500 centistoke, more preferably from about 20 centistoke to about 200 centistoke, even more preferably from about 20 centistoke to about 50 centistoke, as measured under ambient conditions. Non limiting examples of non-volatile, linear silicones suitable for use in the antiperspirant emulsions include but are not limited to, Dow Corning 200, Rhodorsil Oils 70047 available from Rhone-Poulenc, Masil SF Fluid available from Mazer, Dow Corning 225, Dow Corning 1732, Dow Corning 5732, Dow Corning 5750 (available from Dow Corning Corp.); SF-96, SF-1066 and SF18(350) Silicone Fluids (available from G.E. Silicones); Velvasil and Viscasil (available from General Electric Co.); and Silicone L-45, Silicone L530, Silicone L-531 (available from Union Carbide), and Siloxane F-221 and Silicone Fluid SWS-101 (available from SWS Silicones).

Other silicone fluids suitable for use herein include modified or organofunctional silicone carriers such as polyalkylsiloxanes, polyalkyarylsiloxanes, cross-linked silicone elastomers, polyestersiloxanes, polyethersiloxane copolymers, polyfluorosiloxanes, polyaminosiloxanes, and combinations thereof. These modified silicone carriers are typically liquid under ambient conditions, and have a preferred viscosity of less than about 100,000 centistokes, more preferably less than about 500 centistokes, even more preferably from about 1 centistoke to about 50 centistokes, and most more preferably from about 1 centistoke to about 20 centistokes. These modified silicone carriers are generally known in the chemical arts, some examples of which are described in 1 Cosmetics, Science and Technology 27-104 (M. Balsam and E. Sagarin ed. 1972); U.S. Pat. No. 4,202,879, issued to Shelton on May 13, 1980; U.S. Pat. No. 5,069,897, issued to Orr on Dec. 3, 1991; which descriptions are incorporated herein by reference.

The antiperspirant emulsions of the present invention may further comprise other hydrophobic materials, including various solids, non-silicone materials and various volatile hydrophobic materials, for incorporation into the hydrophobic external phase, provided that such other materials are compatible with the other ingredients in the composition and do not unduly impact the stability of the selected emulsion system.

### Emulsifying Agent

The antiperspirant emulsions of the present invention preferably further comprise an emulsifying agent to provide enhanced emulsion stability. The emulsifying agent includes any surfactant that is known for or otherwise compatible with aluminum-containing antiperspirant actives and any other ingredients in the selected formulation. The emulsifying agent may be solubilized in either of the internal and/or external phase, or in any other intermediate phase, but is preferably solubilized or dispersed primarily in the external hydrophobic phase.

Non-limiting examples of suitable emulsifying agents include cationic surfactants, nonionic surfactants, or any other ampholytic surfactant that at the formulation or use pH does not contain a charged cationic species. Preferred emulsifying agents include silicone polyethers such as dimethicone copolyols. These preferred dimethicone copolyols are dimethicone chains with pendant or terminal polyether chains comprising ethylene oxide and propylene oxide, specific examples of which include those copolyols from Dow Corning (DC5225, DC 3225, DC5202, DC5200); General Electric (SF1528); and Witco (Silwet L7622).

Still other suitable emulsifying agents include polysorbitans, fatty acid esters, sorbitan esters, polyglycerol esters, PEG esters, PPG esters, and combinations thereof.

### Optional Ingredients

The antiperspirant emulsions of the present invention may further comprise other optional ingredients to modify the physical, chemical, cosmetic or aesthetic characteristics of the compositions or serve as additional “active” components when deposited on the skin. The compositions may also further comprise optional inert ingredients. Many such optional ingredients are known for use in deodorants, antiperspirants or other personal care compositions, and may also be used in the antiperspirant compositions herein, provided that such optional ingredients are compatible with the other ingredients in the antiperspirant emulsion and do not unduly impact the stability of the selected emulsion system.

Nonlimiting examples of optional ingredients include preservatives, deodorant antimicrobials, fragrances, deodorant perfumes, coloring agents or dyes, thickeners, pH modifiers, pharmaceutical actives, vitamins, and combinations thereof.

Optional ingredients can be in the form of solids or liquids, although the antiperspirant emulsions of the present invention are preferably substantially free of solid materials. In this context, the term “substantially free” means that the emulsions may contain such materials but preferably contain less than 5%, more preferably less than 2%, most preferably zero percent, by weight of such solid materials in the emulsions.

### Method of Use

The antiperspirant emulsions of the present invention may be applied topically to the axilla or other area of the skin in an amount effective to treat or reduce perspiration wetness and/or malodor. The composition is preferably applied in an amount ranging from about 0.1 gram to about 20 grams, more preferably from about 0.1 gram to about 10 grams, even more preferably from about 0.1 gram to about 1 gram, to the desired area of the skin. The compositions are preferably applied to the axilla or other area of the skin, one or two times daily, preferably once daily, to achieve effective antiperspirant and/or malodor control over an extended period. The emulsions applied by such methods may be dispensed from any known liquid dispensing package or device, preferably from a roll-on applicator.

### Method of Manufacture

The antiperspirant emulsions of the present invention may be prepared by any known or otherwise effective technique, suitable for making and formulating a two-phase or other multi-phase emulsion containing an internal polar phase and an external hydrophobic phase. These compositions are generally prepared by formulating an anhydrous emulsion from all of the product ingredients under appropriate shear to form the desired emulsion formulation.

For example, the hydrophobic external phase can be prepared to include all hydrophobic ingredients and any emulsifying agent that is soluble in or otherwise inherently partitions into the hydrophobic. The polar internal phase is then prepared, most typically as a solution, in a separate vessel that includes all polar ingredients such antiperspirant active and any emulsifying agent that is soluble in or otherwise inherently partitions into the polar phase. The polar phase is then added slowly to the external phase under shear to create a uniform emulsified particle size. The resulting anhydrous antiperspirant emulsion is then placed in a suitable application device or container, preferably a roll-on applicator device.

Preferred compositions and methods of manufacture are described hereinafter in the following exemplified embodiments of the antiperspirant emulsions of the present invention.

### EXAMPLES

The following examples further describe and demonstrate specific embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, unless otherwise specified.

The compositions described below (Examples A-D) are anhydrous antiperspirant emulsions containing an internal polar-phase containing solubilized antiperspirant active and a polar solvent, and an external hydrophobic phase containing a non-volatile silicone fluid. Each of the compositions is packaged in a roll-on applicator and are applied to the underarm area of the skin by the methods described herein to provide antiperspirant and deodorant efficacy. The products result in little or no visible residue on the skin and provide for a smooth, dry, non-sticky skin feel during and after application.

Each of the compositions described below (Examples A-D) is prepared by mixing together under shear all of the external phase materials and then mixing together in a separate container all of the internal phase materials until a clear solution forms. The internal phase materials are then added slowly to the external phase materials under sufficient shear to create uniform emulsified particle size. The resulting liquid anhydrous antiperspirant emulsion is then placed in a roll-on applicator package.

TABLE 1
Anhydrous Antiperspirant Emulsions

| Ingredient | A | B | C | D |
|---|---|---|---|---|
| INTERNAL PHASE | | | | |
| Aluminum chlorohydrate solution | | | 20 | 30 |
| (20% active, 40% 1,2 hexanediol, 40% | | | | |
| glycerin) | | | | |
| Aluminum zirconium trichlorohydrate | 20 | 20 | | |
| glycine solution (20% active, 40% 1,2 | | | | |
| hexanediol, 40% glycerin) | | | | |
| Ethanol | 5 | 5 | 5 | 5 |
| EXTERNAL PHASE | | | | |
| Dimethicone 10 cst | 15 | 15 | 15 | 10 |
| Dimethicone (DC1184) | | 59 | | |
| Dimethicone copolyol (DC5225) | 1.0 | 1.0 | 1.0 | 1.0 |
| Cyclopentasiloxane | 59 | | | 30 |
| Hexamethyldisiloxane | | | 59 | |
| Cyclohexasiloxane | | | | 24 |

What is claimed is:

1. Anhydrous antiperspirant emulsions, comprising: (a) an external phase containing a non volatile hydrophobic liquid; and (b) an internal phase containing an antiperspirant active solubilized in a polar solvent;
wherein the emulsions have an external to internal phase ratio of at least about 1.25 and a weight ratio of the non volatile liquid to the combination of antiperspirant active and polar solvent in the internal phase of at least about 0.33.

2. The anhydrous emulsion of claim 1, wherein the phase ratio is from about 1.7 to about 10.

3. The anhydrous emulsion of claim 1, wherein the weight ratio of the non volatile liquid to the combination of antiperspirant active and polar solvent in the internal phase is from about 0.5 to about 3.0.

4. The anhydrous emulsion of claim 3, wherein the phase ratio is from about 1.7 to about 10.

5. The anhydrous emulsion of claim 1 wherein the emulsion comprises from about 5% to about 70% by weight of the non volatile liquid, from about 1% to about 45% by weight of the polar solvent, and from about 0.1% to about 26% by weight of the solubilized antiperspirant active.

6. The anhydrous emulsion of claim 1 wherein the emulsion comprises from about 8% to about 45% by weight of the non volatile liquid, from about 5% to about 35% by weight of the polar solvent, and from about 2% to about 10% by weight of the solubilized antiperspirant active.

7. The anhydrous emulsion of claim 5 wherein the antiperspirant active is an aluminium-containing active, a zirconium-containing active, or combinations thereof.

8. The anhydrous emulsion of claim 6 wherein the antiperspirant active represents from about 0.1% to 20% by weight of the composition and is selected from the group consisting aluminum chlorohydrate, aluminum dichlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum zirconium trichlorohydrate, aluminum zirconium tetrachlorohydrate, aluminum zirconium pentatchlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, aluminum chloride, aluminum sulfate buffered, and combinations thereof.

9. The anhydrous emulsion of claim 5, wherein the internal polar phase further comprises a liquid polyol selected from the group consisting of 1,2-butanediol; 1,2-pentanediol; 4-methyl-1,2-pentanediol; 2-methyl-1,2-pentanediol; 3,3-methyl-1,2-butanediol; 4-methyl-1,2-hexanediol; 1,2-heptanediol; 3-phenyl-1,2-propanediol; 1,2,6-hexanetriol; 1,2-hexandiol; 1,2,4-butanetriol; glycerol isopropyl ether; glycerol propyl ether; glycerol ethyl ether; glycerol methyl ether; glycerol butyl ether; glycerol isopentyl ether; diglycerol isopropyl ether; diglycerol isobutyl ether; diglycerol; triglycerol; triglycerol isopropyl ether; acetic acid glycerol ester; propyleneglycol; polypropyleneglycol; propanoic acid glycerol ester; butanoic acid glycerol ester; 3-methyl butanoic acid glycerol ester; and 3-trimethylsily-1,2-propane diol; and combinations thereof.

10. The anhydrous emulsion of claim 1, wherein the non volatile liquid comprises a silicone-containing liquid at a concentration of from about 0.1% to about 20% by weight of the anhydrous emulsion.

11. The anhydrous emulsion of claim 1, wherein the silicone-containing liquid is a non-volatile dimethicone fluid.

12. The anhydrous emulsion of claim 1 wherein the emulsion further comprises an emulsifying agent.

13. The anhydrous emulsion of claim 12 wherein the emulsifying agent is a silicone-containing emulsifying agent.

14. The anhydrous emulsion of claim 5 wherein the emulsion contains less than 5% by weight of water.

15. The anhydrous emulsion of claim 5 wherein the emulsion contains less than 20% by weight of ethanol.

16. The anhydrous emulsion of claim 15 wherein the emulsion contains less than 3% by weight of ethanol.

17. The anhydrous emulsion of claim 5 wherein the emulsion is visibly clear or translucent.

18. The anhydrous emulsion of claim 5, wherein the emulsion is contained within a roll-on applicator container.

19. The anhydrous emulsion of claim 5, wherein the external phase further comprises cyclopentasiloxane.

20. The anhydrous emulsion of claim 1, wherein the polar solvent comprises glycerin.

21. The anhydrous emulsion of claim 20, wherein the polar solvent further comprises a polyol solvent other than and in addition to the glycerin, wherein the polyol solvent has at least 3 carbon atoms and adjacent hydroxy-substituted carbon atoms at the α and β positions of the polyol solvent.

22. The anhydrous emulsion of claim 21, wherein the polyol solvent other than and in addition to the glycerin is selected from the group consisting of 1,2-butanediol; 1,2-pentanediol; 4-methyl-1,2-pentanediol; 2-methyl-1,2-pentanediol; 3,3-methyl-1,2-butanediol; 4-methyl-1,2-hexanediol; 1,2-heptanediol; 3-phenyl-1,2-propanediol; 1,2,6-hexanetriol; 1,2-hexandiol; 1,2,4-butanetriol; glycerol isopropyl ether; glycerol propyl ether; glycerol ethyl ether; glycerol methyl ether; glycerol butyl ether; glycerol isopentyl ether; diglycerol isopropyl ether; sorbitol; diglycerol isobutyl ether; diglycerol; triglycerol; triglycerol isopropyl ether; acetic acid glycerol ester; propanoic acid glycerol ester; butanoic acid glycerol ester; polypropylene glycol; propylene glycol; 3-methyl butanoic acid glycerol ester; and 3-trimethylsily-1,2-propane diol; and combinations thereof.
